(19) United States
(12) Patent Application Publication
(10) Pub. No.: US 2007/0122399 A1
(43) Pub. Date: May 31, 2007

(54) Composition and method for facilitating bone healing

(76) Inventors: Shrirang Netke, San Bruno, CA (US); Aleksandra Niedzwiecki, San Jose, CA (US); Matthias Rath, Capetown (ZA); Waheed Roomi, Sunnyvale, CA (US); Vadim Ivanov, Castro Valley, CA (US)

Correspondence Address: KENYON & KENYON LLP, ONE BROADWAY, NEW YORK, NY, 10004, US

(21) Appl. No.: 10/570,735

(22) PCT Filed: Jun. 24, 2004
(86) PCT No.: PCT/EP04/06841

§ 371 Date: Dec. 6, 2006

(30) Foreign Application Priority Data
Sep. 5, 2003 (US) 10657019

(51) Int. Cl. A61K 38/43; A61K 31/4415; A61K 31/401; A61K 31/198; A61K 31/525; A61K 31/51; A61K 33/34; A61K 33/04; A61K 33/32; A61K 36/752

(52) U.S. Cl. 424/94.1; 424/638; 424/643; 424/641; 424/702; 424/736; 514/350; 514/423; 514/474; 514/564; 514/251; 514/276; 514/168; 514/52; 424/722

(57) ABSTRACT

The present invention provides a nutritional composition effective in facilitating bone healing in human, comprising lysine, proline, ascorbic acid, copper, vitamin B₆. The nutritional composition contains 27-34% wt lysine, 14-16% wt proline, and 42-47% wt ascorbic acid. The nutritional composition further comprises vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, carnitine, coenzyme Q₁₀, and pycnogenol. The present invention also relates to uses of the nutritional composition in human for facilitating bone healing.

The present invention generally relates to nutritional compositions for facilitating bone healing and uses thereof.

Bone is a dynamic living tissue and is continuously being replenished by resorption and deposition of bone matrix. The stability of bone depends upon the underlying connective tissue. Oxlund H. et al. has shown that optimally structured collagen is more important for bone strength than bone compactness and its calcium saturation (Bone 1996; 19:479-84). A concentration of cross-links between collagen strands appeared 30% less in bone affected by osteoporosis. Knot L. et al. has shown that collagen structure and spatial organization of its fiber network is critical for deposition of minerals and compactness in the bone, and that the micro-architecture of collagen determines bone strength (Bone 1998; 22:181-7). Savvas M. et al. has shown that a loss of collagen caused by malnutrition is a major factor in loss of bone mass (J. Obstetr. Gynecol. 1989; 96:1392-4). The anorexic women in the study show a lowered bone density of 18% in the spine and 25% in the femur. The decrease in bone mass is associated with a 22% decrease in skin collagen.

The healing process after bone fracture is an orderly process that involves multiple phases including: i) hematoma formation; ii) fibro-cartilaginous callus formation; iii) bony callus formation; and iv) bone remodeling. During the healing process, pluripotential cells in the vicinity of the bone fracture differentiate into osteoblasts and chondrocytes. Osteoblasts origin form osteoid tissues and they lay down collagen fibers. Chondrocytes give rise to hypertrophic chondrocytes that deposit a mineralized matrix to form calcified cartilage, which is then remodeled into compact bone. Despite advances in orthopaedic techniques, healing of bone fractures is a lengthy process, often requires weeks if not months. A patient often suffers a severe restriction of movement for several weeks. Facilitating bone healing and fracture repair (i.e., reducing healing time) would be a great relief to the patient. This is particularly desirable in adolescents because this age group of individuals has the lowest compliance with doctor recommendations. When doctors recommend that an adolescent use crutches for a certain period of time, they often remove themselves from crutches much earlier than recommended.

U.S. Pat. No. 6,258,778 discloses a method of enhancing bone and cartilage repair by administering angiotensin and its analogues. U.S. Pat. No. 5,502,074 discloses a method of facilitating bone healing using benzothiophenes. The safety of use of these drugs are not established. For example, angiotensin is known to exert potent cardiovascular and renal effects, and its use in patients with heart or renal failure may be limited.

U.S. Pat. No. 6,061,597 discloses the application of resonant frequency stimulation to promote fracture healing. U.S. Pat. No. 6,290,714 discloses a low level laser therapy in treating bone fracture. The effectiveness of these approach is not shown and requires expensive medical office visits and/or computer equipment. None of these methods has been clinically proven.

U.S. Pat. No. 5,232,709 discloses a nutritional supplement having a large dose of calcium in treating bone loss. Administering to a bone fractured individual with a large dose of calcium would cause mineralization of the bone tissue, rather than supplementing bone collagen. The increased bone mineralization causes further hardening of bone. The affected bone becomes more brittle over time, making it prone to compound fractures and shattering under stress.

There is a long felt need to provide a safe, convenient, affordable and effective approach to facilitate bone healing (i.e., reduce healing time of bone fractures) in humans.

Thus, the technical problem underlying the present invention must be seen as the provision of means and methods to comply with this need. This technical problem is solved by the embodiments characterized in the claims.

Accordingly, the present invention relates to a nutritional composition comprising lysine, proline, ascorbic acid, copper, and vitamin B₆. The nutritional composition is suitable for human use and is effective in facilitating bone healing. The nutritional composition is also suitable for animal use. Preferably, said animal is mammal, most preferably a dog, cat or horse.

Preferably, the nutritional composition contains 27-34% wt lysine, 14-15% wt proline, and 42-47% wt ascorbic acid.

Preferably, the nutritional composition is administered orally. Preferably, the recommended amount is 1,010 mg-8 gram lysine, 560 mg-4 gram proline, 1,500 mg-9 gram ascorbic acid, 2 μg-6 mg copper, and 0.5 mg-10 mg vitamin B₆. More preferably, a recommended amount is 230 mg-10 gram lysine, 120 mg-5 gram proline, 360 mg-15 gram ascorbic acid, 1.5 μg-20 mg copper, and 0.2 mg-20 mg vitamin B₆. Most preferably, a recommended amount is 1,010 mg lysine, 560 mg proline, 1,500 mg ascorbic acid, 330 μg copper and 10 mg vitamin B₆.

Preferably, the nutritional composition is a daily dosage (based on a human subject of average body weight of 72 kg) of 3.2-139 mg/kg lysine, 1.7-69.4 mg/kg proline, 5-208.3 mg/kg ascorbic acid, 0.02-278 μg/kg copper, 2.78-279 μ/kg vitamin B₆.

More preferably, the nutritional composition is a daily dosage of 14-111 mg/kg lysine, 7.8-55.6 mg/kg proline, 20.8-125 mg/kg ascorbic acid, 0.03-83.3 μg/kg copper, and 6.94-139 μg/kg vitamin B₆.

Most preferably, the nutritional composition is a daily dosage of 14 mg/kg lysine, 7.8 mg/kg proline, 20.8 mg/kg ascorbic acid, 4.6 μg/kg copper, 139 μg/kg vitamin B₆.

Preferably, the nutritional composition further comprises vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, carnitine, coenzyme Q₁₀, and pycnogenol.

Preferably, the recommended amount is 67 μg -100 mg vitamin A, 0.7 μg-50 μg vitamin D₃, 0.7 μg-50 μg vitamin E, 1.4 mg-8 mg vitamin B₁, 1.4 mg-8 mg vitamin B₂, 9 mg-250 mg niacin, 18 μg-500 μg folic acid, 4 μg-100 μg vitamin B₁₂13 μg-400 μg biotin, 8 mg-100 mg pantothenic acid, 7 mg-40 mg calcium, 3 mg-300 mg phosphorus, 40 mg-200 mg magnesium, 0.5 mg-10 mg zinc, 20 μg-300 μg selenium, 0.8 mg-15 mg manganese, 2 μg-200 μg chromium, 0.8 μg-100 μg molybdenum, 4 mg-300 mg potassium, 20 mg-500 mg citrus fruit peel bioflavanoids, 10 mg-500 mg arginine, 10 mg-400 mg cysteine, 5 mg-400 mg inositol, 5 mg-400 mg carnitine, 1.6 mg-70 mg coenzyme Q₁₀, and 1.6 mg-70 mg pycnogenol.

More preferably, the recommended amount is 166 μg-50 mg vitamin A, 1.65 μg-20 μg vitamin D₃, 1.65 μg-20 μg vitamin E, 3.5 mg-7 mg vitamin B₁, 3.5 mg-7 mg vitamin B₂, [symbol] 5 mg-100 mg niacin, 45 μg-300 μg folic acid, 10 μg-50 μg vitamin B₁₂, 32 μg-300 [symbol] biotin, 20 mg-60 mg pantothenic acid, 17 mg-35 mg calcium, 7 mg-100 mg phosphorus, 50 mg-100 mg magnesium, 3 mg-8 mg zinc, 30 μg-250 μg selenium, 1 mg [symbol] 0.25 mg manganese, 2 μg-75 μg chromium, 2 μg-75 μg molybdenum, 8 mg-200 mg potassium, 50 mg -250 mg citrus fruit peel bioflavanoids, 100 mg -300 mg arginine, 80 mg [symbol] .00 mg cysteine, 80 mg-200 mg inositol, 80 mg-200 mg camitine, 3 mg-35 mg enzyme Q₁₀, and 3 mg-35 mg pycnogenol.

Most preferably, the recommended amount is 333 μg vitamin A, 3.3 μg vitamin D₃, 3.3 μg vitamin E, 7 mg vitamin Bi, 7 mg vitamin B₂, 45 mg niacin, 90 μg folic acid, 20 μg vitamin [symbol] , 65 μg biotin, 40 mg pantothenic acid, 35 mg calcium, 15 mg phosphorus, 40 mg magnesium, 7 mg zinc, 20 μg selenium, 1.3 mg manganese, 10 μg chromium, 4 μg [symbol] polybdenum, 20 mg potassium, 100 mg citrus fruit peel bioflavanoids, 40 mg arginine, 35 mg [symbol] teine, 35 mg inositol, 35 mg camitine, 7 mg coenzyme Q₁₀, and 7 mg pycnogenol.

Preferably, the nutritional composition comprises a daily dosage (based on a human subject of average body weight of 72 kg) of 0.9-1,390 μg/kg vitamin A, 0.01-0.694 μg/kg vitamin D₃, [symbol] 1-0.694 μg/kg vitamin E, 19.4-111 μg/kg vitamin B₁, 19.4-111 μg/kg vitamin B₂, 125-3,472 [symbol] kg niacin, 0.25-6.94 μg/kg folic acid, 0.05-1.39 μg/kg vitamin B₁₂, 0.181-5.56 μg/kg [symbol] tin, 111-1,390 μg/kg pantothenic acid, 97.2-555 μg/kg calcium, 42-4,167 μg/kg phosphorus, 555-2,778 μg/kg magnesium, 6.9-139 μg/kg zinc, 0.28-4.17 μg/kg selenium, [symbol] 1-208.3 μg/kg manganese, 0.03-2.78 μg/kg chromium, 0.01-1.39 μg/kg molybdenum, [symbol] 6-4,167 μg/kg potassium, 278-6.944 μg/kg citrus fruit peel bioflavanoids, 139-6,944 μg/kg [symbol] inine, 135-5,555 μg/kg cysteine, 69-5,555 μg/kg inositol, 69-5,555 μg/kg carnitine, 22.2-2 μg/kg coenzyme Q₁₀, and 22.2-972 μg/kg pycnogenol.

More preferably, the nutritional composition comprises a daily dosage (based on a human object of average body weight of 72 kg) of 2.31-694 μg/kg vitamin A, 0.023-0.278 μg/kg vitamin D₃, 0.023-0.278 μg/kg vitamin E, 48.6-97.2 μg/kg vitamin B₁, 48.6-97.2 μg/kg vitamin B₂, 312.5-3,190 μg/kg niacin, 0.6-4.17 μg/kg folic acid, 0.14-0.69 μg/kg vitamin B₁₂, [symbol] .44-4.17 μg/kg biotin, 278-833 μg/kg pantothenic acid, 236-903 μg/kg calcium, 97.2-1,390 μg/kg phosphorus, 694-1,390 μg/kg magnesium, 41.7-111 μg/kg zinc, 0.42-3.47 μg/kg [symbol] enium, 13.9-45.1 μg/kg manganese, 0.07-2.78 μg/kg chromium, 0.03-1.04 μg/kg molybdenum, 111.1-2,778 μg/kg potassium, 694-3,472 μg/kg citrus fruit peel bioflavanoids, 1,389-4,167 μg/kg arginine, 1,111-2,778 μg/kg cysteine, 1,111-2,778 μg/kg inositol, 1,111-2,778 μg/kg camitine, 41.7-486 μg/kg coenzyme Q₁₀, and 41.7-486 μg/kg pycnogenol. Most preferably, the nutritional composition comprises a daily dosage (based on a human subject of average body weight of 72 kg) of 4.6 μg/kg vitamin A, 0.046 μg/kg vitamin D₃, 0.046 μg/kg vitamin E, 97.2 μg/kg vitamin B₁, 97.2 μg/kg vitamin B₂, 625 μg/kg niacin, 1.25 μg/kg folic acid, 0.27 μg/kg vitamin B₁₂, 0.9 μg/kg biotin, 555 μg/kg pantothenic acid, 486 μg/kg calcium, 208 μg/kg phosphorus, 555 μg/kg magnesium, 97.2 μg/kg zinc, 0.78 μg/kg selenium, 18.1 μg/kg manganese, 0.14 μg/kg chromium, 0.06 μg/kg molybdenum, 277.8 μg/kg potassium, 1,389 μg/kg citrus fruit peel bioflavanoids, 555 μg/kg arginine, 486 μg/kg cysteine, 486 μg/kg inositol, 486 μg/kg carnitine, 97.2 μg/kg coenzyme Q₁₀, and 97.2 μg/kg pycnogenol. 0.01-0.694 μg/kg vitamin D₃, 18.1 μg/kg manganese, 555 μg/kg arginine, 486 μg/kg cysteine, 4.6 μg/kg vitamin A, 0.046 μg/kg vitamin E, 97.2 μg/kg vitamin B₁, 97.2 μg/kg vitamin B₂, 0.27 μg/kg vitamin B₁₂, 625 μg/kg niacin, 1.25 μ/kg folic acid, 0.9 μg/kg biotin, 555 μg/kg pantothenic acid, 486 μg/kg calcium, 208 μg/kg phosphorus, 555 μg/kg magnesium, 97.2 μg/kg zinc, 0.78 μg/kg selenium, 0.14 μg/kg chromium, 0.06 μg/kg molybdenum, 277.8 μg/kg potassium, 1,389 μg/kg citrus fruit peel bioflavanoids, 486 μg/kg inositol, 486 μg/kg carnitine, 97.2 μg/kg coenzyme Q₁₀, and 97.2 μg/kg pycnogenol.

The present invention provides a method for facilitating bone healing in a mammal, comprising the step of administering to a mammal in need thereof an effective amount of a nutritional composition comprising lysine, proline, ascorbic acid, copper, and vitamin B₆.

The present invention further provides a method for facilitating bone healing in a mammal comprises the step of administering to a mammal in need thereof of an effective amount of a nutritional composition further comprises vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, carnitine, coenzyme Q₁₀, and pycnogenol.

Preferably, the mammal is a human. preferably, the nutritional composition is effective in reducing healing time for bone fractures. preferably, the healing time is reduced>about 5%. More preferably, the healing time is reduced>about 15%. Most preferably, the healing time is reduced>about 50%.

Preferably, the nutritional composition is effective in human of all ages. Preferably, the nutritional composition is suitable for facilitating bone healing in adults of 41-40 and 41-50 years of age. The nutritional composition provides a 37% and 40% reduction in healing time respectively. More preferably, the nutritional composition is effective in human of 10-20 years of age (i.e., adolescents), which provides a 49% reduction in healing time.

Preferably, the nutritional composition may be administered orally, intravenously, or [symbol] arenterally.

As used herein, the term “bone healing” refers to the healing of bone fractures. Bone healing [symbol] all also encompass the process of bone repair and shall not be limited to healing of accidental bone fractures. Bone healing also concerns surgical intervention of bones such as bone replacement (e.g., hip and knee joint replacement) and bone implantation (e.g., tooth implantation). When a bone is healed, the normal mobility at the fractured bone site is restored and there is absence of pain elicited by stressing the fracture or by walking and [symbol] eneral restoration of efficient and painless functioning of the affected limb at the fracture site.

The term “healing time” refers to the time elapsed from the time when bone fracture occurs until the time when the bone fracture is healed. With respect to the experiments performed in the studies disclosed herein, the healing time is measured for the time elapsed from the time of reduction of fractured bone until the bone is healed. “Reduction” refers to the process of aligning the tips of a fractured bone (e.g., tibia) at the point of fracture in a position to allow using of the fractured bone tips together. “Adolescent” is a human between about 10 and about 20 years of age. “Effective amount” refers to an amount of the present nutritional composition effective in reducing the healing time of bone fracture. “Pharmaceutically acceptable” refers to carriers, diluents, and excipients that are compatible with the other ingredients of the formulation, and not deleterious to the recipient thereof. “% wt” refers to % of a specific ingredient as a % proportion to the total weight of the nutritional composition,

For example, 27% wt of lysine refers to a nutritional composition in which 27% of the total height of the nutritional composition is lysine.

The present nutritional composition is suitable for use in a mammal. Preferably, the mammal [symbol] a human. Different age groups of human may exhibit different speeds of bone healing. For example, human of elder age may be easier to suffer from bone fracture (due to [symbol] calcification by osteoporosis) and is likely to have a longer healing time. The present nutritional composition is found to be effective in faciliating bone healing in human in mineral; and not particularly limited to a particular age group.

The present invention provides a nutritional composition for facilitating bone healing in human, preferably in adolescent individuals, comprising the step of administering to a human [symbol] need of treatment an effective amount of the composition comprising lysine, proline, [symbol] corbic acid, copper, and vitamin B₆ Preferably, the nutritional composition contains 27-34% wt. ysine, 4247% wt ascorbic acid and 14-15% wt proline.

The present nutritional composition also contains lysine and proline. Lysine and proline are constituents of collagen and proteins in the bone. Lysine and proline may contribute to [symbol] teoblast proliferation of alkaline phosphatase, nitric oxide, insulin like growth factor-I, and collagen type I and may be essential for proper bone formation.

Lysine may include lysine salts such as hydroxylysine and hydroxylysine salts. A daily dose of 3.2-139 mg/kg lysine is recommended. Preferably, 14 to 111 mg/kg lysine is used; and More preferably, 14 mg/kg lysine is used. For an average individual weighing 72 kg, the daily recommended dosage of lysine is 230 mg to 10 grams; preferably, 1,010 mg to 8 grams; and more preferably 1,010 mg.

Proline is a non-essential amino acid. However, its synthesis in human body could be limited under certain conditions. It has been reported that the stress of fracture lowers non-essential amino acid levels in plasma of elder humans. In such a case, deficiency of proline, a semi-essential amino acid, if any, would adversely affect the healing of fracture, since this amino acid is present in a large proportion in collagen.

Proline may include proline salts such as hydroxyproline and hydroxyproline salts. A daily dose of 1.7-69.4 mg/kg proline is recommended. Preferably, 7.8 to 56 mg/kg is used; and

More preferably, 7.8 mg/kg is used. For an average individual weighing 72 kg, the daily recommended dosage of proline is 120 mg to 5 grams; preferably, 560 mg to 4 grams; and more preferably 560 mg.

The present nutritional composition contains ascorbic acid. Ascorbic acid may promote the progressive development of osteoblast phenotype and facilitate bone healing and it is also necessary for the differentiation and proliferation of osteogenic and chondrogenic cells.

Ascorbic acid and vitamin C are used interchangeably. The term ascorbic acid encompasses ascorbic acid and salts thereof. Preferably, the ascorbic acid to be applied in accordance with the present invention is calcium ascorbate, magnesium ascorbate or ascorbyl palmitate. A daily dose of 5-208 mg/kg ascorbic acid is recommended. Preferably, 20.8-125 mg/kg is used; and more preferably, 20.8 mg/kg is used. For an average individual weighing 72 kg, the daily recommended dosage of ascorbic acid is 360 mg to 15 grams; preferably, 1,500 mg to 9 grams; and more preferably 1,500 mg.

The present invention further provides a nutrient composition further comprising minerals and/or trace elements. Trace elements may help to catalyze the production of these macromolecules needed for connective tissue structure and function. Preferred trace elements in accordance with the present invention are iron, iodine, copper, zinc, manganese, cobalt, [symbol] lybdenum, selenium, chromium, nickel, tin, fluorine or vanadium.

Copper, as a cofactor for lysyl oxidase, is essential for intra- and intermolecular cross-links in collagen. Copper deficit has been shown to impair the mechanical strength of bone. It was hypothesized that a relatively large quantity of ascorbic acid, vitamin B₆, L-lysine and L-proline, together with copper, would have a pronounced effect on bone collagen health and function to produce a marked difference in healing time between fractured bones of a supplement group and a placebo group.

Copper compounds may include copper glycinate. A daily dose of 0.02 to 278 μg/kg copper is recommended. Preferably, 0.03 to 83 μg/kg is used; and more preferably, 4.6 μg/kg is used. For an average individual weighing 72 kg, the daily recommended dosage of 1.5 μg to 20 mg; preferably 2 μg to 6 mg; and more preferably, 330 μg.

Vitamin B₆ is of importance in bone healing, as it is instrumental in providing reducing equivalents necessary for mineralization. Vitamin B₆ deficiency caused marked diminution in glucose 6-phosphate dehydrogenase activity in perisoteal bone formation and in developing callus. It also caused changes in the bone suggestive of imbalance between osteoblasts and osteoclasts.

Vitamin B₆ compounds may include pryridosine HCl. A daily dose of 2.8 to 279 μg/kg vitamin B₆ is recommended. Preferably, 7 to 139 μg/kg vitamin B₆ is used; and more preferably, 139 μg/kg is used. For an average individual weighing 72 kg, the daily recommended dosage of vitamin B₆ is 0.2 to 20 mg; preferably, 0.5 to 10 mg; and more preferably, 10 mg.

Certain ingredients of the nutritional composition according to the invention are present at a high amount. Specifically, lysine is present between 27-34% wt (preferably at 28-33% wt); proline is present between 14-16% wt (preferably 15-16% wt); and ascorbic acid is present between 42-47% wt (preferably at 4346% wt).

Unexpectedly, it has been found in accordance with the present invention that a nutritional composition as specified herein efficiently facilitates bone healing (i.e. it reduces the healing time) of bone fractures in humans, particularly in adolescents. Advantageously, administration of the nutritional composition of the present invention does not merely facilitate bone healing efficiently, but also improves general well being, and is cost effective.

A recommended daily oral dosage includes 3.2-139 mg/kg lysine, 1.37-69 mg/kg proline, 5-208 mg/kg ascorbic acid, 2.78-279 μg/kg vitamin B₆, and 0.02-278 μg/kg copper. Preferably, the recommended daily oral dosage is: 14-111 mg/kg lysine, 7.8- 56 mg/kg proline, 20.8-125 mg/kg ascorbic acid, 6.9-139 μg/kg vitamin B₆, and 0.03-83 μg/kg copper. More preferably, the recommended daily oral dosage is: 14 mg/kg lysine, 7.8 mg/kg proline, 20.8 mg/kg ascorbic acid, 139 μg/kg vitamin B₆, 4.6 μg/kg copper. Preferably, the nutritional composition is administered 3 tablets per day (i.e., one tablet in morning, one tablet in afternoon and one tablet at night).

Several other dietary components, such as: protein, calcium, magnesium, zinc, copper, iron, fluoride, and vitamins D, A and K, are required for normal bone metabolism. All of these nutrients impact fracture healing, some more directly than others. The trauma of bone fracture was shown to cause a decrease in copper, manganese, and zinc levels in liver, suggesting increased requirement of these minerals after bone fracture.

The present nutritional composition may further comprise vitamin D₃, manganese, arginine, cysteine, vitamins A, E, B₁, B₂, B₁₂, niacin, folic acid, biotin, pantothenic acid, calcium, phosphorus, magnesium, zinc, selenium, chromium, molybdenum, potassium, citrus fruit peel bioflavanoids, inositol, carnitine, coenzyme Q₁₀, and pycnogenol.

The particular dosage of the present nutritional composition required to facilitate bone healing (i.e., reducing healing time) will depend on the severity of the medical condition, the route of administration and the particular subject being treated. The nutritional composition of the present invention may be administered by a variety of routes including oral, intravenous, or parenteral administration. Preferably, the nutritional composition is in unit dosage form, e.g. tablets or capsules. Thus, the present nutritional composition is recommended to be administered as an orally tablet preparation.

A daily recommended dosage (based on a human subject of average body weight of 72 kg) may further contain 0.9-1,390 μg/kg vitamin A, 0.01-0.694 μg/kg vitamin D₃, 0.01-0.694 μg/kg vitamin E, 19.4-111 μg/kg vitamin Bi, 19.4-111 μg/kg vitamin B₂, 125-3,472 μg/kg niacin, 0.25-6.94 μg/kg folic acid, 0.05-1.39 μg/kg vitamin B₁₂, 0.181-5.56 μg/kg biotin, 111-1,390 μg/kg pantothenic acid, 97.2-555 μg/kg calcium, 424,167 μg/kg phosphorus, 555-2,778 μg/kg magnesium, 6.9-139 μg/kg zinc, 0.28-4.17 μg/kg selenium, 11.1-208.3 μg/kg manganese, 0.03-2.78 μg/kg chromium, 0.01-1.39 μ/kg molybdenum, 55.6-4,167 μg/kg potassium, 278-6.944 μg/kg citrus fruit peel bioflavanoids, 139-6,944 μg/kg arginine, 135-5,555 μg/kg cysteine, 69-5,555 μg/kg inositol, 69-5,555 μg/kg carnitine, 22.2-972 μg/kg coenzyme Q₁₀, and 22.2-972 μg/kg pycnogenol.

Preferably, the daily recommended dosage (based on a human subject of average body weight of 72 kg) may further contain 2.31-694 μg/kg vitamin A, 0.023-0.278 μg/kg vitamin D₃, 0.023-0.278 μg/kg vitamin E, 48.6-97.2 μg/kg vitamin B₁, 48.6-97.2 μg/kg vitamin B₂, 312.5-3,190 μg/kg niacin, 0.6-4.17 μ/kg folic acid, 0.14-0.69 μg/kg vitamin B₁₂, 0.444-4.17 μg/kg biotin, 278-833 μg/kg pantothenic acid, 236-903 μg/kg calcium, 97.2-1,390 μg/kg phosphorus, 694-1,390 μg/kg magnesium, 41.7-111 μg/kg zinc, 0.42-3.47 μg/kg selenium, 13.9-45.1 μg/kg manganese, 0.07-2.78 μg/tkg chromium, 0.03-1.04 μg/kg molybdenum, 111.1-2,778 μg/kg potassium, 694-3,472 μg/kg citrus fruit peel bioflavanoids, 1,389-4,167 μg/kg arginine, 1,111-2,778 μg/kg cysteine, 1,111-2,778 μg/kg inositol, 1,111-2,778 μg/kg [symbol] rnitine, 41.7486 μg/kg coenzyme Q₁₀, and 41.7486 μg/kg pycnogenol.

Most preferably, the nutritional composition may further include a daily dosage (based on a human subject of average body weight of 72 kg) of 4.6 μg/kg vitamin A, 0.046 μg/kg vitamin D₃, 0.046 μg/kg vitamin E, 97.2 μg/kg vitamin Bi 97.2 μg/kg vitamin B₂, 625 μg/kg niacin, [symbol] .25 μg/kg folic acid, 0.27 μg/kg vitamin B₁₂, 0.9 μg/kg biotin, , 555 μg/kg pantothenic acid, [symbol] 86 μg/kg calcium, 208 μg/kg phosphorus, 555 μg/kg magnesium, 97.2 μg/kg zinc, 0.78 μg/kg selenium, 18.1 μg/kg manganese, 0.14 μg/kg chromium, 0.06 μg/kg molybdenum, [symbol] 77.8 μg/kg potassium, 1,389 jig/kg citrus fruit peel bioflavanoids, 555 μg/kg arginine, 486 μg/kg cysteine, 486 μg/kg inositol, 486 μg/kg camitine, 97.2 μg/kg coenzyme Q₁₀, and 97.2 μg/kg pycnogenol. 0.01-0.694 μg/kg vitamin D₃, 18.1 μg/kg manganese, 555 μg/kg arginine, 86 μg/kg cysteine, 4.6 μg/kg vitamin A, 0.046 μg/kg vitamin E, 97.2 μg/kg vitamin B₁, 97.2 μ/kg vitamin B₂, 0.27 μg/kg vitamin B₁₂, 625 μg/kg niacin, 1.25 μg/kg folic acid, 0.9 μg/kg biotin, 555 μg/kg pantothenic acid, 486 μg/kg calcium, 208 μg/kg phosphorus, 555 μg/kg magnesium, 97.2 μg/kg zinc, 0.78 μ/kg selenium, 0.14 μg/kg chromium, 0.06 μg/kg polybdenum, 277.8 μg/kg potassium, 1,389 μg/kg citrus fruit peel bioflavanoids, 486 μg/kg [symbol] ositol, 486 μg/kg carnitine, 97.2 μg/kg coenzyme Q₁₀, and 97.2 μg/kg pycnogenol.

The present nutritional composition may include a pharmaceutically acceptable carrier, diluent, or excipient. Nutritional composition of the present invention can be prepared by procedures known in the art. Respective ingredients may be formulated with common excipients, diluents, or carriers, and formed into tablets, capsules, suspensions, powders, and the like. Examples of excipients, diluents, and carriers include: i) fillers and extenders such as starch, sugars, mannitol, and silicic derivatives; ii) binding agents such as carboxymethyl cellulose and other cellulose derivatives, alginates, gelatin, and polyvinyl-pyrrolidone; iii) moisturizing agents such as glycerol; disintegrating agents such as calcium carbonate and sodium bicarbonate; agents for retarding dissolution such as paraffin; iv) resorption accelerators such as quaternary ammonium compounds; v) surface active agents such as acetyl alcohol, and glycerol monostearate; v) adsorptive carriers such as kaolin and bentonite; and vi) lubricants such as talc, calcium and magnesium stearate, and solid polyethyl glycols.

The nutritional compositions may also be formulated as elixirs or solutions for convenient oral administration or as solutions appropriate for parenteral administration, for example, by intramuscular, subcutaneous or intravenous routes. Ideally the formulation is in the form of a pill, tablet, capsule, lozenge, liquid or similar dosage form. The nutritional compositions may well be suited to formulation as sustained release dosage forms and the like.

Tablets Preparation

The ingredients listed in Table 1 were formulated to form tablets. The tablets contained the key ingredients of lysine (1,010 mg), proline (560 mg), ascorbic acid (1,500 mg), copper (330 μg) and vitamin B₆ (10 mg).

The tablets further contained additional ingredients including vitamin D₃, manganese, arginine, cysteine, vitamins A, E, B₁, B₂, B₁₂, niacin, folic acid, biotin, pantothenic acid, calcium, phosphorus, magnesium, zinc, selenium, chromium, molybdenum, potassium, citrus fruit peel bioflavanoids, inositol, carnitine, coenzyme Q₁₀, and pycnogenol.

TABLE 1
Serving Size - Three Tablets/Day

| | |
|---|---|
| Daily | Daily Dosage |
| Dosage | per Body Weight* |

| Key Ingredients | | | | |
|---|---|---|---|---|
| L-Lysine (from L-Lysine HCl) | 1,010 mg | | 14.0 | mg/kg |
| | (28% wt) | | | |
| L-Proline | 560 mg | | 7.8 | mg/kg |
| | (16% wt) | | | |
| Ascorbic Acid (Ascorbyl Palmitate, | 1,500 mg | | 20.8 | mg/kg |
| Calcium Ascorbate, Magnesium | (42.8% wt) | | | |
| Ascorbate) | | | | |
| Copper (Copper Glycinate) | 330 μg | | 4.58 | μg/kg |
| | (<0.01% wt) | | | |
| Vitamin B₆ (Pyridoxine HCl) | 10 mg | | 139 | μg/kg |
| | (0.28% wt) | | | |
| Additional Ingredients | | | | |
| Vitamin A (7.5% Betatene (Henkel)) | 333 | μg | 4.6 | μg/kg |
| Vitamin D₃ (Cholecalciferol) | 3.3 | μg | 0.046 | μg/kg |
| Vitamin E (Mixed Covitol) | 3.3 | μg | 0.046 | μg/kg |
| Vitamin B₁ (Thiamine Mononitrate) | 7 | mg | 97.2 | μg/kg |
| Vitamin B₂ (Riboflavin) | 7 | mg | 97.2 | μg/kg |
| Niacin (Niacinamide) | 45 | mg | 625 | μg/kg |
| Folic Acid | 90 | μg | 1.25 | μg/kg |
| Vitamin B₁₂ (Cyanocobalamin) | 20 | μg | 0.27 | μg/kg |
| Biotin | 65 | μg | 0.90 | μg/kg |
| Pantothenic Acid (D-Calcium | 40 | mg | 555 | μg/kg |
| Pantothenate) | | | | |
| Calcium (Gycinate, Ascorbate) | 35 | mg | 486 | μg/kg |
| Phosphorus (Dicalcium Phosphate) | 15 | mg | 208 | μg/kg |
| Magnesium (Magnesium Glycinate, | 40 | mg | 555 | μg/kg |
| Magnesium Ascorbate) | | | | |
| [symbol] inc (Zinc Glycinate) | 7 | mg | 97.2 | μg/kg |
| [symbol] elenium (L-Selenomethionine) | 20 | μg | 0.78 | μg/kg |
| Manganese (Amino Acid Chelate) | 1.3 | mg | 18.1 | μg/kg |
| Chromium (Chromium Glycanate) | 10 | μg | 0.14 | μg/kg |
| Molybdenum (Molybdenum Glycinate) | 4 | μg | 0.06 | μg/kg |
| Potassium (Potassium Proteinate) | 20 | mg | 277.8 | μg/kg |
| Citrus Fruit Peel Bioflavanoids | 100 | mg | 1,389 | μg/kg |
| [symbol] -Arginine (L-Arginine HCl) | 40 | mg | 555 | μg/kg |
| [symbol] -Cysteine (L-Cysteine Monohydrate | 35 | mg | 486 | μg/kg |
| HCl) | | | | |
| [symbol] nositol | 35 | mg | 486 | μg/kg |
| [symbol] -Carnitine (L-Carnitine Tartrate) | 35 | mg | 486 | μg/kg |
| CoEnzyme Q₁₀ | 7 | mg | 97.2 | μg/kg |
| [symbol] ycnogenol | 7 | mg | 97.2 | μg/kg |
| TOTAL | 3.5 | g | 48.6 | mg/kg |

Body Weight refers to a human subject of average body weight of 72 kg

Clinical Studies
Patient Selection:

[symbol] randomized double-blind placebo-controlled study was performed. The clinical study was conducted according to the recommendations of the Declaration of Helsinki, its amendments and AMG. The inclusion criteria for patient admission were: (i) unilateral displaced closed or [symbol] ade I open fractures of tibial shaft; and (ii) age above 10 years. The exclusion criteria for patient admission were: (i) patients who had other major injuries, (ii) patients with cardiopulmonary, rheumatological, neurological or metabolic diseases, (iii) patients with previous injuries which influenced their general functions, (iv) patients with fractures within 5 [symbol] n distal to tibial tuberosity or within 5 cm proximal to the ankle joint.

Admitted patients were either receive standard care and placebo or standard care with supplementation with a nutritional supplement comprising lysine, proline, ascorbic acid, copper and vitamin B₆. Qualifying patients, on admission to the study, were clinically examined and the radiographs of the affected limbs were taken, fractures reduced under anesthesia and above knee plaster casts applied.

Efforts were made to ensure that age groups and fracture types were equally distributed in the [symbol] groups. Patients were entered in the study from February 2001 until December 2002. A total of 113 patients with unilateral displaced closed or grade I open tibial fractures studied. Admitted patients were given informed consent. Participants were advised that data obtained from the studies would be submitted for publication. Out of these, 54 patients were assigned to the supplemented group and 59 patients were assigned to the placebo group. Table 2 summarizes the age distribution of the total 131 patients.

TABLE 2
Distribution of Patients by Age Groups

| | Age | | | | | |
|---|---|---|---|---|---|---|
| | 10-20 | 21-30 | 31-40 | 41-50 | >50 | |
| | years | years | years | years | years | Total |
| Supple- | 2 | 7 | 6 | 3 | 3 | 21 |
| mented | (9.5%)* | (33.3%) | (28.6%) | (14.3%) | (14.3%) | (100%) |
| Group | | | | | | |
| Placebo | 8 | 10 | 10 | 4 | 4 | 36 |
| Group | (22.2%) | (27.8%) | (27.8)% | (11.1%) | (11.1%) | (100%) |

Values in parenthesis represent percentage of total patients in the specified age group

Clinical Protocol:

All fractures were reduced (closed reduction) under anesthesia and above knee plaster casts applied. The fractured limbs were routinely radiographed before and after reduction. The supplemented group of patients were supplied with the nutritional composition in Table 1 and the placebo group of patients with bottles containing placebo tablets. The nutritional composition listed in Table 1 were studied to evaluate if the proposed desired supplements can ensure adequacy of nutrients impacting fracture healing. The placebo tablets contained material of no medical significance, such as cellulose, fructose etc., but were physically [symbol] distinguishable from nutrient tablets. All patients were asked to take one tablet thrice daily morning, afternoon and night).

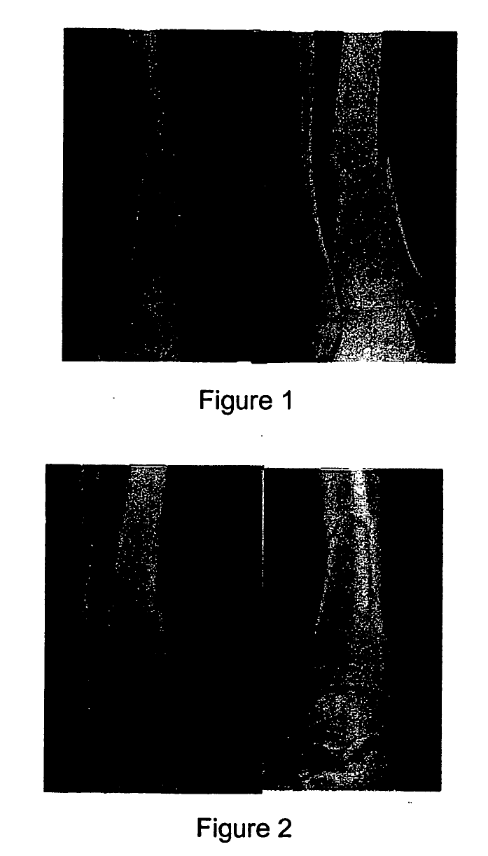

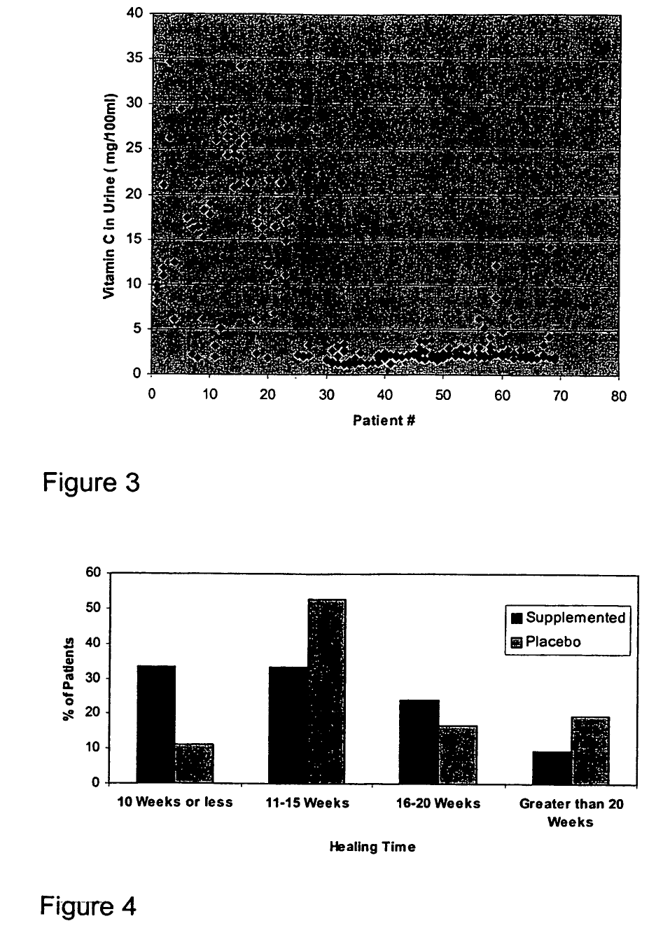

Urine samples of the patients were taken to evaluate their baseline ascorbic acid levels. Blood samples were also taken to assess the baseline calcium levels (FIG. 3). The patients were [symbol] en discharged from the hospital and were asked to return for check-up every four weeks until the treating orthopedic surgeon deemed the fracture healed. At each follow-up examination, tibial fractures were radiographed, and patients were clinically examined and tested for urinary vitamin C and blood calcium levels. The radiological examination was done to confirm that the fragments of the fracture remained in reduced position and that callus formation was progressing satisfactorily. The ascorbic acid content of the urine was determined by spectrophotometry and the blood calcium content was determined with commercial kits. Healing was defined as absence of abnormal mobility at the fracture site clinically and absence of pain elicited by stressing the fracture or by walking. Radiographic confirmation of callus formation was used as supporting evidence for healing. (See FIGS. 1 and 2 for radiographic examples). A healing period of greater than 20 weeks without any surgical intervention was considered delayed healing.

Statistical Analysis

The results were expressed as means±standard error for the groups. The Wilcoxon test was conformed on the fracture healing times among various groups. Statistical significance was that P<0.05.

Results:

The patients from the supplemented group and 42 patients from the placebo group returned for regular follow-ups until fractures were deemed healed.

Overall, the urinary ascorbic acid content in supplemented patient group was higher than that [symbol] the placebo group (FIG. 3). Low urinary ascorbic acid values (below 5 mg/100 ml of [symbol] ine) were detected in eight supplemented patients and high ascorbic acid values (above 5 μg/100 ml of urine) in six placebo patients at any one of the check-up visits. These patients are excluded from evaluation. Therefore, only 21 patients in supplemented group and 36 patients in the placebo group remained for the completion of the study. Blood calcium levels [symbol] all participants in the study were within normal limits.

The age distribution of the patients is detailed in Table 2. The age range of the patients in the supplemented group was between 15 to 65 years, with a mean age of 35 years. The age range [symbol] the patients in the placebo group was between 12 to 75 years, with a mean age of 32 years.

The healing time of the patients is detailed in Table 4. The mean healing time for the patients [symbol] the supplemented group was 14.0±1.1 weeks. The mean healing time for the patients in the placebo group was 16.9±1.2 weeks. These data show that the overall mean healing time in the supplemented group is about three weeks shorter than that for the placebo group (i.e., 17.2 [symbol] ). This difference attain statistical significance at t=1.07, p=0.288.

Percentile classification of the data indicates that in the 75th percentile, fractures in the supplemented group healed within 17 weeks, while those in placebo group healed in 19 weeks Table 3).

TABLE 3
Effect of Supplementation on Bone Fracture Healing Time

| | Supplemented | Placebo |
|---|---|---|
| Criteria | Group | Group |
| Number of patients | 21 | 36 |
| Age range (years) | 15 to 65 | 12 to 75 |
| Mean age (years) | 35 | 32 |
| Healing time (weeks) | 14.0 ± 1.1 | 16.9 ± 1.2 |
| 75^{th} percentile healing period (weeks) | 17 | 19 |

The percentage of patients experiencing early fracture healing (in 10 weeks or less) differed by 33.3% in the supplemented group and 11.1% in the placebo group (Chi-square analysis=2.853, p=0.091). The percentage of patients that had delayed healing (more than 20 weeks) was 9.5% in supplemented group and 19.4% in the placebo group (Table 4).

TABLE 4
Distribution (Percentage) of Patients by Fracture Healing Time

| Healing Time | Supplemented Group | Placebo Group |
|---|---|---|
| 10 weeks or less | 33.3% | 11.1% |
| 11 to 15 weeks | 33.3% | 52.8% |
| 16 to 20 weeks | 23.8% | 16.7% |
| More than 20 weeks | 9.5% | 19.4% |

The healing time of bone fracture in patients of different ages is shown in Table 6. In the patients of 10-20 years of age, the healing time reduced from 17.6 weeks to 9 weeks (reduced [symbol] 49%). In the patients of 31-40 years of age, the healing time reduced from 17.1 weeks to [symbol] 10.7 weeks (reduced 37%). In the patients of 41-50 years of age, the healing time reduced from 21.2 weeks to 12.7 weeks (reduced 40%). In the patients of >50 years of age, the [symbol] fling time reduced from 16 weeks to 15.7 weeks (reduced 1.8%). Overall, the healing time [symbol] the supplemented group, except in the 21-30 year old group, is reduced. (Table 5).

TABLE 5
Healing Time (in Weeks) for Various Age Groups

| | 10-20 | 21-30 | 31-40 | 41-50 | |
|---|---|---|---|---|---|
| [symbol] e Groups | yr | yr | yr | yr | >50 yr |
| [symbol] pplemented Group | | | | | |
| [symbol] aling Time | | | | | |
| [symbol] an (in wks) | 9 | 18.3 | 10.7 | 12.7 | 15.7 |
| [symbol] nge (in wks) | 9 | 9-26 | 6-16 | 11-14 | 14-19 |
| [symbol] cebo Group | | | | | |
| [symbol] aling Time | | | | | |
| [symbol] an (in wks) | 17.6 | 14.6 | 17.1 | 21.2 | 16 |
| [symbol] nge (in wks) | 9-30 | 9-30 | 11-39 | 13-30 | 10-20 |

The data suggest that administering to patients suffering from bone fracture with the present nutritional composition at the specified doses effectively reduces the healing time by at least two weeks in 75% of the patients. Patients in the supplemented group also reported an enhanced feeling of general well being during the study. The strongest effects can be seen in the adolescent age group (i.e., 10 to 20 years of age) who has a 49% reduction in the healing time. Patients who are 41-50 years and 31-40 years of age also have a significant reduction in the healing time (i.e., 40% and 37%, respectively). It is believed that patients in the other age groups may likely to receive the same benefits if the dosage of the nutritional supplementation [symbol] optimized.

There was no signficantly difference in the blood calcium levels in the supplemented group as compared to that in the placebo group. All the patients were found to have their blood calcium levels within the normal range.

The reduction in healing time is believed to be due to the supplementation of key ingredients which comprise lysine, proline, ascorbic acid, copper and vitamin B₆, as well as additional ingredients used in the present study. The present data provide evidence that nutritional supplementation in patients suffering from bone fracture can facilitate the healing (i.e., reduce healing time). Administration of the present nutritional compositions to bone fractured patients would have a positive impact early functional recovery, improved well being, reduced medical costs, and reduced cost to business.

It will be understood that there is no intent to limit the present invention to the preferred embodiment disclosed, but rather it is intended to cover all modifications and alternate constructions falling within the spirit and scope of the invention. All publications and other references mentioned herein are incorporated by reference in their entirety.

The figures show:

FIG. 1 depicts a radiograph of tibial shaft fracture immediately prior to reduction.

FIG. 2 depicts radiograph of tibial shaft fracture at healing at 12 weeks.

FIG. 3 depicts ascorbic acid levels (urinalysis) in supplemented (patient #1-29) and placebo patient #30-70) groups.

FIG. 4 depicts a distribution of patients (percentage) by tibial fracture healing time.

1. A nutritional composition suitable for facilitating bone healing in a mammal, comprising lysine, proline, ascorbic acid, copper, and vitamin B₆.

2. The nutritional composition of claim 1, wherein the nutritional composition contains 27-34% wt lysine, 14-15% wt proline, and 42-47% wt ascorbic acid.

3. The nutritional composition of claim 1, wherein the nutritional composition provides a daily dosage of
a) 230 mg-10 grams lysine, 120 mg-5 grams proline, 360 mg-15 grams ascorbic acid, 1.5 μg-20 mg copper, and 0.2 mg-20 mg vitamin B₆;
b) 1,010 mg-8 grams lysine, 560 mg-4 grams proline, 1,500 mg-9 grams ascorbic acid, 2 μg-6 mg copper, and 0.5 mg-10 mg vitamin B₆; or
c) 1,010 mg lysine, 560 mg proline, 1,500 mg ascorbic acid, 330 μg copper and 10 mg vitamin B₆.

4. The nutritional composition of claim 1, wherein said the nutritional composition provides a daily dosage per body weight of
a) 3.2-139 mg/kg lysine, 1.7-69.4 mg/kg proline, 5-208.3 mg/kg ascorbic acid, 0.02-278 μg/kg copper, and 2.78-279 μg/kg vitamin B₆;
b) 14-111 mg/kg lysine, 7.8-55.6 mg/kg proline, 20.8-125 mg/kg ascorbic acid, 0.03-83.3 μg/kg copper, and 6.94-139 μg/kg vitamin B₆; or
c) 14 mg/kg lysine, 7.8 mg/kg proline, 20.8 mg/kg ascorbic acid, 4.6 μg/kg copper, and 139 μg/kg vitamin B₆.

5. The nutritional composition of claim 1, wherein the nutritional composition further comprises vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, camitine, coenzyme Q₁₀, and pycnogenol.

6. The nutritional composition of claim 5, wherein the nutritional composition provides a daily dosage of
a) 67 μg-100 mg vitamin A, 0.7 μg-50 μg vitamin D₃, 0.7 μg-50 μg vitamin E, 1.4 mg-8 mg vitamin B₁, 1.4 mg-8 mg vitamin B₂, 9 mg-250 mg niacin, 18 μg-500 μg folic acid, 4 μg-100 μg vitamin B₁₂, 13 μg-400 μg biotin, 8 mg-100 mg pantothenic acid, 7 mg-40 mg calcium, 3 mg-300 mg phosphorus, 40 mg-200 mg magnesium, 0.5 mg-10 mg zinc, 20 μg-300 μg selenium, 0.8 mg-15 mg manganese, 2 μg-200 μg chromium, 0.8 μg-100 μg molybdenum, 4 mg-300 mg potassium, 20 mg-500 mg citrus fruit peel bioflavanoids,10 mg-500 mg arginine, 10 mg-400 mg cysteine, 5 mg-400 mg inositol, 5 mg-400 mg camitine, 1.6 mg-70 mg coenzyme Q₁₀, and 1.6 mg-70 mg pycnogenol;
b) 166 μg-50 mg vitamin A, 1.65 μg-20 μg vitamin D₃, 1.65 μg-20 μg vitamin E, 3.5 mg-7 mg vitamin B₁, 3.5 mg-7 mg vitamin B₂, 22.5 mg-100 mg niacin, 45 μg-300 μg folic acid, 10 μg-50 μg vitamin B₁₂, 32 μg-300 μg biotin, 20 mg-60 mg pantothenic acid, 17 mg-35 mg calcium, 7 mg-100 mg phosphorus, 50 mg-100 mg magnesium, 3 mg-8 mg zinc, 30 μg- 250 μg selenium, 1 mg-3.25 mg manganese, 2 μg-75 μg chromium, 2 μg -75 μg molybdenum, 8 mg-200 mg potassium, 50 mg-250 mg citrus fruit peel bioflavanoids, 100 mg-300 mg arginine, 80 mg-200 mg cysteine, 80 mg-200 mg inositol, 80 mg-200 mg camitine, 3 mg-35 mg coenzyme Q₁₀, and 3 mg-35 mg pycnogenol; or
c) 333 μg vitamin A, 3.3 μg vitamin D₃, 3.3 μg vitamin E, 7 mg vitamin B₁, 7 mg vitamin B₂, 45 mg niacin, 90 μg folic acid, 20 μg vitamin B₁₂, 65 μg biotin, 40 mg pantothenic acid, 35 mg calcium, 15 mg phosphorus, 40 mg magnesium, 7 mg zinc, 20 μg selenium, 1.3 mg manganese, 10 μg chromium, 4 μg molybdenum, 20 mg potassium, 100 mg citrus fruit peel bioflavanoids, 40 mg arginine, 35 mg cysteine, 35 mg inositol, 35 mg carnitine, 7 mg coenzyme Q₁₀, and 7 mg pycnogenol.

7. The nutritional composition of claim 5, wherein said composition further comprises the following components in the following daily dosage per body weight
a) 0.9-1,390 μg/kg vitamin A, 0.01-0.694 μg/kg vitamin D₃, 0.01-0.694 μg/kg vitamin E, 19.4-111 μg/kg vitamin B₁, 19.4-111 μg/kg vitamin B₂, 125-3,472 μg/kg niacin, 0.25-6.94 μg/kg folic acid, 0.05-1.39 μg/kg vitamin B₁₂, 0.181-5.56 μg/kg biotin, 111-1,390 μg/kg pantothenic acid, 97.2-555 μg/kg calcium, 42-4,167 μg/kg phosphorus, 555-2,778 μg/kg magnesium, 6.9-139 μg/kg zinc, 0.28-4.17 μg/kg selenium, 11.1-208.3 μg/kg manganese, 0.03-2.78 μg/kg chromium, 0.01-1.39 μg/kg molybdenum, 55.6-4,167 μg/kg potassium, 278-6.944 μg/kg citrus fruit peel bioflavanoids, 139-6,944 μg/kg arginine, 135-5,555 μg/kg cysteine, 69-5,555 μg/kg inositol, 69-5,555 μg/kg carnitine, 22.2-972 μg/kg coenzyme Q₁₀, and 22.2-972 μg/kg pycnogenol;
b) 2.31-694 μg/kg vitamin A, 0.023-0.278 μg/kg vitamin D₃, 0.023-0.278 μg/kg vitamin E, 48.6-97.2 μg/kg vitamin B₁, 48.6-97.2 μg/kg vitamin B₂, 312.5-3,190 μg/kg niacin, 0.6-4.17 μg/kg folic acid, 0.14-0.69 μg/kg vitamin B₁₂, 0.444-4.17 μg/kg biotin, 278-833 μg/kg pantothenic acid, 236-903 μg/kg calcium, 97.2-1,390 μg/kg phosphorus, 694-1,390 μg/kg magnesium, 41.7-111 μg/kg zinc, 0.42-3.47 μg/kg selenium, 13.9-45.1 μg/kg manganese, 0.07-2.78 μg/kg chromium, 0.03-1.04 μg/kg molybdenum, 111.1-2,778 μg/kg potassium, 694-3,472 μg/kg citrus fruit peel bioflavanoids, 1,389-4,167 μg/kg arginine, 1,111-2,778 μg/kg cysteine, 1,111-2,778 μg/kg inositol, 1,111-2,778 μg/kg carnitine, 41.7-486 μg/kg coenzyme Q₁₀, and 41.7-486 μg/kg pycnogenol; or
c) 4.06 μg/kg vitamin A, 0.046 μg/kg vitamin D₃, 0.046 μg/kg vitamin E, 97.2 μg/kg vitamin B₁, 97.2 μg/kg vitamin B₂, 625 μg/kg niacin, 1.25 μg/kg folic acid, 0.27 μg/kg vitamin B₁₂, 50.9 μg/kg biotin,, 555 μg/kg pantothenic acid, 486 μg/kg calcium, 208 μg/kg phosphorus, 555 μg/kg magnesium, 97.2 μg/kg zinc, 0.78 μg/kg selenium, 18.1 μg/kg manganese, 0.14 μg/kg chromium, 0.06 μg/kg molybdenum, 277.8 μg/kg potassium, 1,389 μg/kg citrus fruit peel bioflavanoids, 555 μg/kg arginine, 486 μg/kg cysteine, 486 μg/kg inositol, 486 μg/kg camitine, 97.2 μg/kg coenzyme Q₁₀, and 97.2 μg/kg pycnogenol.

8. The nutritional composition of claim 1, wherein the mammal is a human.

9. A pharmaceutical composition comprising the nutritional composition of claim 1.

10. A method of facilitating bone healing in a mammal comprising administering to the mammal the pharmaceutical composition of claim 9.

11. The method of claim 10, wherein the mammal is a human.

12. The method of claim 10, wherein the pharmaceutical composition is administered orally, intravenously or parenterally.
